(19) United States
(12) Patent Application Publication
(10) Pub. No.: US 2007/0286825 A1
(43) Pub. Date: Dec. 13, 2007

(54) Cosmetic agents and methods for the preparation thereof

(76) Inventor: Kenneth E. Visek, Stickney, IL (US)

Correspondence Address: OLSON & HIERL, LTD., 20 NORTH WACKER DRIVE, 36TH FLOOR, CHICAGO, IL, 60606, US

(21) Appl. No.: 11/665,790

(22) PCT Filed: Nov. 2, 2005
(86) PCT No.: PCT/US05/39658

§ 371 Date: Apr. 18, 2007

Related U.S. Application Data
(60) Provisional application No. 60/624,706, filed on Nov. 3, 2004.

(51) Int. Cl. A61K 8/49

(52) U.S. Cl. 424/59; 549/403

(57) ABSTRACT

A cosmetic agent is disclosed comprising a cosmetically useful group bound to an amino functional group by a linking moiety. The linking moiety comprises an alkylidene or an arylidene group bearing two carboxyl groups. The cosmetically useful group is bound to one carboxyl group of the linking moiety, while the amino functional group is bound to the other carboxyl group of the linking moiety. The amino functional group comprises an amino substituent bound to a carbon atom of an alkylidene group having an oxygen substituent, and the oxygen substituent is bound to the other carboxyl group of the linking moiety by an ester bond. The amino substituent can be a primary amino substituent, a secondary amino substituent, a tertiary amino substituent, or a quaternary amino substituent. Preferably the amino substituent is a tertiary or a quaternary amino substituent. Methods for preparing, and compositions comprising, cosmetic agents of this invention are also disclosed.

### CROSS-REFERENCE TO RELATED APPLICATION

This application claims the priority of U.S. Provisional Application for Patent Ser. No. 60/624,706 filed on Nov. 3, 2004 which is incorporated herein by reference.

### FIELD OF THE INVENTION

This invention relates to cosmetic agents. More particularly, this invention relates to cosmetic agents which are covalently linked to an amino functional group.

### BACKGROUND OF THE INVENTION

Cosmetics and cosmeceutical preparations typically contain a wide array of materials including cosmetic ingredients that impart discernible cosmetic or sensory benefits to the skin and hair, such as antioxidants, vitamins, provitamins, coenzymes, essential oils, skin tanning agents, sunscreen agents, conditioners, moisturizers, emollients, and the like. Other cosmetic ingredients include substantially inert ingredients such as fillers, thickeners, fragrances, colorants, and the like, which are used to enhance the physical properties of the finished product. Many materials used in cosmetics can serve multiple functions (i.e., can both enhance the physical properties of the product and impart some cosmetic or sensory benefit to the skin or hair).

Frequently, it is desirable to retain a cosmetic agent on the skin or hair after washing or rinsing the skin or hair to which the cosmetic agent has been applied to prolong the residual cosmetic or sensory benefit over some period of time. Many cosmetic agents, however, are not very substantive for skin and hair, and tend to wash off relatively easily. There is an ongoing need for cosmetic agents with improved substantivity to skin and/or hair. The present invention fulfills this need.

### SUMMARY OF THE INVENTION

A cosmetic agent of the present invention comprises a cosmetically useful group bound to an amino functional group by a linking moiety. The linking moiety comprises an alkylidene or an arylidene group bearing two carboxyl groups. The cosmetically useful group is bound to the one carboxyl group of the linking moiety, while the amino functional group is bound to the other carboxyl group of the linking moiety. The amino functional group comprises an amino substituent bound to a carbon atom of an alkylidene group having an oxygen substituent, and the oxygen substituent is bound to the other carboxyl group of the linking moiety by an ester bond. The amino substituent can be a primary amino substituent, a secondary amino substituent, a tertiary amino substituent, or a quaternary amino substituent. In the case of primary, secondary, and tertiary amino substituents, the amino substituent can be present in the form of a free base or as a salt. Preferred salts are mineral acid salts, such as chlorides, bromides, sulfates, phosphates, and the like, as well as organic acid salts, such as acetates, propionates, lactates, citrates, maleates, succinates, and the like. Preferably, the amino substituent is a tertiary or a quaternary amino substituent. Preferably, the linking moiety is a dicarboxylated C₁-C₁₈ alkylidene, a dicarboxylated C₆-C₁₀ arylidene, or a dicarboxylated C₁-C₁₈ alkyl-substituted C₆-C₁₀ arylidene.

A preferred quaternized cosmetic agent embodiment of the present invention comprises a compound of formula (I): 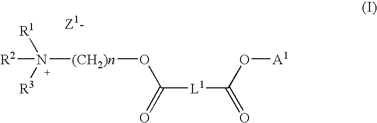
in which R¹, R², and R³ are each independently selected from the group conisting of C₁-C₁₈ alkyl, C₁-C₁₈ alkenyl, and benzyl; n is an integer having a value in the range of 2 to about 18; L¹ is a divalent organic radical selected from the group consisting of C₁-C₁₈ alkylidene, C₆-C₁₀ arylidene, and C₁-C₁₈ alkyl-substituted C₆-C₁₀ arylidene; Z¹ is a halide ion, a C₁-C₁₈ alkylsulfate ion, a C₁-C₁₈ alkylsulfonate, a benzylsulfate ion, a benzylsulfonate ion, or an arylsulfonate ion; and A¹ is a physiologically tolerable, cosmetically useful group. Preferably, A¹ is an antioxidant, a vitamin, a provitamin, a coenzyme, an essential oil, a skin tanning agent, and/or a sunscreen agent.

The quaternized cosmetic agents of the present invention are substantive to keratinaceous materials, such as skin, hair and nails, providing a means for depositing a physiologically tolerable, cosmetically useful material thereon in a manner that resists rinse off. The quaternized cosmetic agents of the present invention also can impart conditioning properties to skin and hair, in particular. The ester linkage between the cosmetically useful group and the remainder of the quaternized molecule can provide for controlled release of a cosmetically useful material over time under some conditions, depending on the nature of the cosmetically useful group in the quaternized cosmetic agent.

In a method aspect of the present invention, a quaternized cosmetic agent is prepared as shown in Scheme 1 condensing an amino alcohol having the formula (II) and a hydroxyl-substituted, physiologically tolerable, cosmetically useful material (A) with a dicarboxylic acid or dicarboxylic anhydride linking group (L) to form a compound of formula (III). The compound of formula (III) is then quaternized by reaction with an alkylating agent (R³-Z¹) to afford a quaternized cosmetic agent of formula (I). 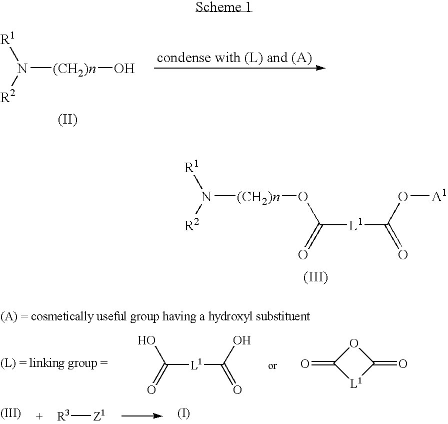
In each of formulas (I), (II), and (III), the groups R¹, R², R³, L¹, Z¹, A¹ and n are each as defined above.

A preferred tertiary amino-substituted cosmetic agent embodiment of the present invention is a compound of formula (III), or a salt thereof, as described above.

The cosmetic agents of the present invention are substantive to keratinaceous substances, such as skin, hair, and the like, and provide a means for depositing a physiologically tolerable, cosmetically useful material on the skin, hair, or other keratinaceous substrate in a manner that resists rinse off, for example.

### DETAILED DESCRIPTION OF PREFERRED EMBODIMENTS

The term “cosmetic agent” and grammatical variations thereof, as used herein and in the appended claims, refers to cosmetically useful materials that are applied to external keratinous surfaces of the human body, including the skin, hair and nails. For convenience, and not by way of limitation, the invention is described in terms of applications to the skin and/or hair of humans, but can encompass non-human creatures, such as pets and avian species where grooming products are similarly employed on fur, feathers, beaks, and/or claws.

The phrase “physiologically tolerable”, as used herein and in the appended claims in reference to cosmetically useful materials, means that the materials are capable of topical administration to or contacting human skin without the production of undesirable physiological effects, such as irritation, itching, stinging, or systemic effects such as nausea, dizziness, and the like.

The term “antioxidant”, as used herein and in the appended claims, refers to materials that can inhibit or suppress oxidation reactions and/or free radical reactions, including such reactions as may occur on the skin, hair, or other external surfaces of the body.

The term “vitamin”, as used herein and in the appended claims, refers to known low molecular weight organic compounds that are required in trace amounts for normal growth and metabolic processes. The term “provitamin” refers to an organic compound that can be converted to a vitamin by a natural reaction in an organism.

The term “coenzyme” (also known as an “enzyme cofactor”), as used herein and in the appended claims, refers to an organic compound that interacts with and generally binds to an enzyme and is necessary for activation or deactivation of the enzyme.

The phrase “essential oil”, as used herein and in the appended claims, refers to an oil obtained from a natural source, typically a plant source, used for its flavor, fragrance and/or chemically active properties, and includes mixtures of compounds as well as purified components.

The phrase “skin tanning agent”, as used herein and in the appended claims, refers to materials that promote or enhance the visible tanning of human skin, including artificial skin tanning materials.

The phrase “sunscreen agent”, as used herein and in the appended claims, refers to materials that are capable of protecting the skin and hair from ultraviolet (UV) radiation, such as UVA radiation (light having a wavelength in the range of about 320 to about 400 nm) and UVB radiation (light having a wavelength in the range of about 290 to about 320 nm).

The term “cosmetically useful”, as used herein and in the appended claims, when used in reference to a material or a portion of a quaternized cosmetic agent means that the material or portion can enhance discernible, aesthetic properties, such as appearance or tactile perception, of external keratinous portions of the body, including the skin, hair, and nails. Non-limiting examples of cosmetically useful materials and groups include antioxidants such as butylated hydroxytoluene (BHT), ascorbic acid (vitamin C), and alpha-tocopherol (vitamin E), vitamins such as vitamin A, vitamin D, vitamin C, and vitamin E, provitamins such as panthenol (provitamin B5), coenzymes such as coenzyme Q10 (ubiquinone) or reduced forms thereof such as ubiquinol, essential oils such as sandalwood oil, and patchouli alcohol, skin tanning agents such as dihydroxyanthroquinone (DHA), sunscreen agents, such as benzophenones, para-aminobenzoic acid (PABA), and esters of PABA, as well as colorants, humectants, emollients, and like materials that are recognized by persons of ordinary skill in the cosmetic arts as useful for inclusion in a cosmetic formulation. Non-limiting examples of desirable properties imparted by cosmetically useful materials include, coloration, wrinkle abatement, smoothness, suppleness, sheen, gloss, moisturization, deodorization, combability, barrier properties, fragrance, protection from oxidation (i.e., antioxidant properties), protection from sun (i.e., sunscreen properties), amelioration of aging damage, and the like.

As used herein and in the appended claims, the term “Cₓ to C_{y}” or “Cₓ-C_{y}” in reference to an alkyl group, an aryl group or any other chemical moiety, means that the moiety includes about x to about y carbon atoms in its chemical formula, where x an y are positive integers. For example, C₂ to C₄ alkyl means an alkyl group selected from ethyl, propyl, isopropyl, butyl, sec-butyl, isobutyl or tert-butyl. The term “substituted” in reference to a chemical moiety such as an alkyl group or cosmetically useful group, means that the alkyl group or cosmetically useful group includes one or more specified substituents along the alkyl chain. For example, hydroxy-substituted C₂-C₃ alkyl includes 1-hydroxyethyl, 2-hydroxyethyl, 1,2-dihydroxyethyl, 1-hydroxypropyl, 1,2-dihydroxypropyl, 1,2,3-trihydroxypropyl, 2-hydroxypropyl, and the like.

The phrase “divalent radical” or the suffix “ylidine” as used herein and in the appended claims in reference to a chemical moiety or group means that the moiety or group has two bonds to other chemical groups or has two positions open for formation of chemical bonds to other groups. For example “alkylidene” means an aliphatic hydrocarbon moiety having two bonding positions available, whereas “C₆ to C₁₀ arylidene” means a phenyl of naphthyl moiety having two positions open for bonding to other groups.

The cosmetic agents of the invention comprise a physiologically tolerable, cosmetically useful group bound by a linking moiety to an amino functional group. The cosmetically useful group can be any substance that has utility in a cosmetic formulation. Preferably, the cosmetically useful group is selected from the group consisting of an antioxidant, a vitamin, a provitamin, a coenzyme, an essential oil, a skin tanning agent, and/or a sunscreen agent.

The linking moiety comprises two carboxyl groups tethered together by an alkylidene or arylidene group. Preferably, the linking moiety is a dicarboxylated C₁-C₁₈ alkylidene, a dicarboxylated C₆-C₁₀ arylidene, or a dicarboxylated C₁-C₁₈ alkyl-substituted C₆-C₁₀ arylidene. The linking moiety is bound to the cosmetically useful group by an ester bond with one of its carboxyl substituents. The linking moiety is also bound to the amino functional group by an ester bond with its other carboxyl substituent.

The amino functional group comprises an amino substituent bound to an alkylidene group having an oxygen substituent, the oxygen substituent being bound to the other carboxyl group of the linking moiety. Preferably the oxygen substituted alkylidene group is a C₂-C₁₈ alkyloxy group. The amino substituent can be a primary amino, a secondary amino, a tertiary amino, or a quaternary amino substituent.

A preferred quaternized cosmetic agent of the present invention comprises a compound of formula (I): 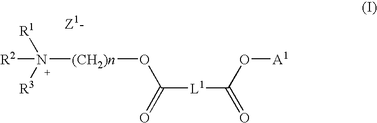
In formula (I), the groups R¹, R², and R³ are each independently selected from the group consisting of C₁-C₁₈ alkyl, C₁-C₁₈ alkenyl, and benzyl; n is an integer having a value in the range of 2 to about 18, and are bound to a the nitrogen of an amino alkyl ester, forming a quaternary ammonium moiety. A¹ is a physiologically tolerable, cosmetically useful group. A linking group consists of a tether L¹ attached to two carboxyl moieties which connect the cosmetically useful group and the quaternary ammonium moiety together by ester bonds. L¹ is a divalent organic radical selected from the group consisting of C₁-C₁₈ alkylidene, C₆-C₁₀ arylidene and C₁-C₁₈ alkyl-substituted C₆-C₁₀ arylidene. The counter ion, Z¹ is a halide ion (e.g., a chloride or bromide ion), a C₁-C₁₈ alkylsulfate ion (e.g., a methosulfate or ethosulfate ion), a C₁-C₁₈ alkylsulfonate ion (e.g., a methylsulfonate ion), a benzylsulfate ion, a benzylsulfonate ion, and an arylsulfonate ion (e.g., a toluenesulfonate ion, a phenyl sulfonate ion).

Any physiologically tolerable, cosmetically useful group (A¹) can be included in the quaternized cosmetic agent of the present invention so long as it can be bonded to the linking group via an ester bond. Preferably, A¹ is selected from the group consisting of an antioxidant, a vitamin, a provitamin, a coenzyme, an essential oil, a skin tanning agent, and a sunscreen agent. Particular cosmetically useful groups may be classified in more than one category, for example, alpha-tocopherol is both an antioxidant, and a vitamin.

A particularly preferred quaternized cosmetic agent of the present invention is a quaternary-substituted tocopherol compound having the formula (IV): 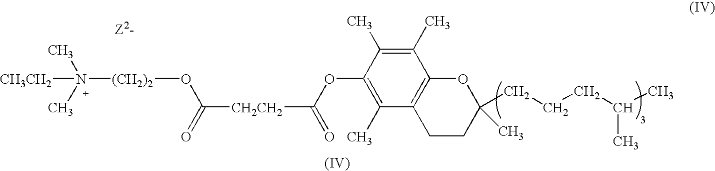
wherein Z² is a halide ion, a C₁-C₁₈ alkylsulfate ion, a C₁-C₁₈ alkylsulfonate ion, a benzylsulfate ion, a benzylsulfonate ion, or an arylsulfonate ion. Preferably Z² is a halide ion or a C₁-C₁₈ alkylsulfate ion, more preferably; Z² is a chloride, bromide or ethosulfate ion, most preferably ethosulfate ion.

In a preferred method aspect, a quaternized cosmetic agent is prepared by condensing an amino alcohol having the formula (II) and a hydroxy-substituted, physiologically tolerable, cosmetically useful material with a dicarboxylic acid or dicarboxylic anhydride linker to form a compound of formula (III). The compound of formula (III) is then quaternized by reaction with an alkylating agent (R³-Z¹) to afford a quaternized cosmetic agent of formula (I), as shown in Scheme 2. The linker comprises tether L¹ attached to two carboxylic acid groups or the cyclic anhydride thereof. 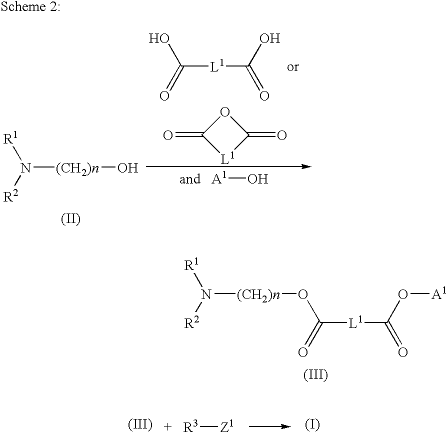
In formulas (I), (II), and (III), the groups R¹, R², and R³ are each independently selected from the group consisting of C₁-C₁₈ alkyl, C₁-C₁₈ alkenyl, and benzyl; n is an integer having a value in the range of 2 to about 18; L¹ is divalent organic radical selected from the group consisting of C₁-C₁₈ alkylidene, C₆-C₁₀ arylidene and C₁-C₁₈ alkyl-substituted C₆-C₁₀ arylidene; Z¹ is selected from the group consisting of a halide ion, a C₁-C₁₈ alkylsulfate ion, a C₁-C₁₈ alkylsulfonate, a benzylsulfate ion, a benzylsulfonate ion, and an arylsulfonate ion; and A¹ is a physiologically tolerable, cosmetically useful group. Preferably, A¹ is an antioxidant, a vitamin, a provitamin, a coenzyme, an essential oil, a skin tanning agent, and/or a sunscreen agent. Particularly preferred is a vitamin moiety such as vitamin E (alpha tocopherol).

A preferred tertiary amino-substituted cosmetic agent of the present invention is a compound of formula (III), or a salt thereof: 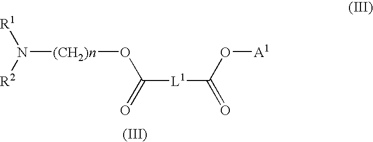
wherein R¹ and R² are each independently selected from the group consisting of C₁-C₁₈ alkyl, C₁-C₁₈ alkenyl, and benzyl; n is an integer having a value in the range of 2 to about 18; L¹ is a divalent organic radical selected from the group consisting of C₁-C₁₈ alkylidene, C₆-C₁₀ arylidene and C₁-C₁₈ alkyl-substituted C₆-C₁₀ arylidene; and A¹ is a physiologically tolerable, cosmetically useful group. Preferred slats include mineral acid salts, such as salts of hydrochloric acid, sulphuric acid, phosphoric acid, and the like, as well as salts of organic acids, such as acetic acid, propionic acid, lactic acid, citric acid, succinic acid, and the like.

The cosmetic agents of the present invention preferably are substantive to keratinaceous materials, such as hair, wool, skin, and nails, providing a means for depositing a physiologically tolerable, cosmetically useful material, such as an antioxidant, a vitamin, or a provitamin thereon in a manner that resists rinse off, for example. The cosmetic agents of the present invention also afford substantive conditioning properties to the skin and hair. The ester linkages between the physiologically tolerable, cosmetically useful group and the remainder of the quaternized molecule can provide for controlled release of a cosmetically useful material over time under some conditions, depending on the nature of the cosmetically useful moiety group in the molecule, for example, by ester hydrolysis over a period of time.

The following examples are intended to illustrate preferred embodiments, but not limit the present invention.

### EXAMPLE 1

Preparation of Quaternized Tocopherol, Procedure A

About 458 parts by weight (1.03 molar equivalents) of alpha tocopherol (synthetic vitamin E) was heated at a temperature in the range of about 120 to about 125° C. together with one part by weight of toluenesulfonic acid and about 97 parts by weight (0.97 molar equivalents) of succinic anhydride to produce tocopherol monosuccinate ester. The reaction was monitored by periodically determining the acid value and by infrared (IR) spectrometry. After about 2 hours, the acid value dropped from a value of about 200 to an acid value of about 115, and the IR spectrum indicated the presence of two carbonyl absorption peaks at about 1711 and 1753 reciprocal centimeters (cm⁻¹), indicating a substantially complete reaction.

The resulting tocopherol monosuccinate was heated at a temperature in the range of about 150 to about 155° C. with about 110 parts by weight of dimethylaminoethanol, added portionwise, until the acid value dropped to about 20 and the IR spectrum showed only one broad carbonyl band at about 1737 cm⁻¹. The resulting tertiary amino-substituted cosmetic agent is a compound of formula (III) in which the amino substituent is a tertiary amino group, i.e., dimethylamino, and the cosmetically useful group is a vitamin, i.e., tocopherol. This dimethyamino-substituted tocopherol was then dissolved in about 189 parts by weight of polyethylene glycol-400 (PEG-400), and quaternized by addition of about 154 parts by weight of diethylsulfate over a period of about one hour at a temperature in the range of about 58 to about 62° C., until the free amine content, as determined by titration, was less than about 1% (active basis) to afford compound (IV) in which Z² is ethosulfate ion. The resulting solution of Compound (IV) ethosulfate in PEG-400, (referred to herein as IV/PEG), had an acid value of less than about 20, a water content of 1% or less and a free amine content of 1% or less, on a weight basis. 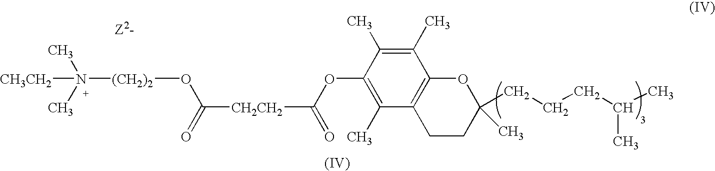
wherein Z² is an ethosulfate ion.

### EXAMPLE 2

Preparation of Quaternized Tocopherol, Procedure B

About 495 parts by weight of alpha tocopherol (synthetic vitamin E) was heated at a temperature in the range of about 125 to about 130° C. together with 0.8 parts by weight of toluenesulfonic acid and 108 parts by weight succinic anhydride to produce tocopherol monosuccinate ester. The reaction was monitored by periodically determining the acid value and by infrared (IR) spectrometry. After about 3 hours, the acid value dropped from about 210 down to about 114 (preferably the acid value is in the range of about 106 to about 118), and the IR spectrum indicated the presence of two carbonyl absorption peaks at about 1711 and 1753 reciprocal centimeters (cm⁻¹), indicating a substantially complete reaction.

About 2 parts by weight of hypophosphorous acid and about 112 parts by weight dimethylaminoethanol were added to the resulting tocopherol monosuccinate. The mixture was heated to, and maintained at, a temperature in the range of about 155 to about 160° C., under a nitrogen purge, and small amounts of dimethylaminoethanol were added to the reaction mixture until the acid value dropped below about 20 and the amine value stabilized at about 95 (preferably the amine value is in the range of about 84 to about 101). The IR spectrum of the product showed only one broad carbonyl band at about 1737 cm⁻¹. The resulting ester was then cooled to a temperature in the range of about 58 to about 62° C. and the cooled ester was dissolved in about 153 parts by weight of polyethylene glycol-400 (PEG-400). About 167 parts by weight of diethylsulfate was then slowly added to quaternize the ester. The temperature of the reaction mixture was maintained at a range of about 58 to about 62° C., until the free amine content, as determined by titration, was less than about 1% (active basis) to afford Compound (IV) ethosulfate. The resulting solution of Compound (IV) ethosulfate had an acid value of less than about 20, a water content of 1% or less, a free amine content of 1% or less, and comprised about 85% Compound (IV) ethosulfate, on a solution weight basis.

Additionally, Compound (IV) ethosulfate was prepared by procedure B using natural vitamin E in place of the synthetic material. Other solutions of Compound (IV) ethosulfate were prepared by substantially the same procedure, except that the quaternized ester was dissolved in an alternative solvent, such as, butylene glycol (1,3-butanediol), mineral oil, or vitamin E, for example, instead of PEG-400.

A solution containing about 85% of Compound (IV) ethosulfate prepared with butylene glycol as the solvent by the described procedure is referred to herein as IV/BG. A solution containing about 85% of Compound (IV) ethosulfate prepared with vitamin E as the solvent by the described procedure is referred to herein as IV/VE.

### EXAMPLE 3

Substantivity to Keratin of A Quaternized Cosmetic Agent of the Invention.

The substantivities to keratin, such as wool, hair and skin, of compositions containing Compound (IV) ethosulfate, prepared by Procedure B of Example 2, were determined by a modification of the well-known cationic sorption test commonly called “Rubine Dye Test.” (See, for example, Crawford, et al., “A replacement for Rubine dye for detecting cationics on keratin,” J. Soc. Cosmet. Chem., V3 1, pp. 273-278 (September/October 1980), the relevant disclosures of which are incorporated by reference).

The compositions A-H, listed in Table 1, were prepared with solutions of IV/BG and IV/VE, of Example 2 (as supplied), containing about 85% Compound (IV) ethosulfate and 15% solvent, added in the amounts indicated. Compositions A, B, E, and F each represents about 0.1 active weight % of Compound (IV) ethosulfate, and Compositions C, D, G, and H each represents about 0.5 active weight % of Compound (IV) ethosulfate.

TABLE 1
Compositions Containing Compound (IV) Ethosulfate
INGREDIENTS (% BY WEIGHT AS SUPPLIED)

| | IV/BG | IV/VE | | | | |
|---|---|---|---|---|---|---|
| | (85/15) | (85/15) | Cetearyl | TWEEN ® 20 | Water:IPA | Water |
| Comp. | of Ex. 2 | of Ex. 2 | Alcohol | (30%) | (9:1) | Deionized |
| A | 0.13 | — | — | — | Q.S. to 100% | — |
| B | — | 0.12 | — | — | Q.S. to 100% | — |
| C | 0.63 | — | — | — | Q.S. to 100% | — |
| D | — | 0.59 | — | — | Q.S. to 100% | — |
| E | 0.13 | — | 8 | 5 | — | Q.S. to 100% |
| F | — | 0.12 | 8 | 5 | — | Q.S. to 100% |
| G | 0.63 | — | 8 | 4 | — | Q.S. to 100% |
| H | — | 0.59 | 8 | 4 | — | Q.S. to 100% |

TWEEN ® 20 is the trade name for polysorbate 20, sold by ICI Surfactants.

Compositions A-D were prepared by dissolving the IV/BG or IV/VE ingredient in a hydroalcoholic medium having a weight ratio of 9 parts water (deionized): 1 part isopropanol (IPA). Emulsion compositions E-H were prepared by heating the cetearyl alcohol and water to a temperature of about 60° C., until homogeneous, adding the TWEEN® 20 ingredient, stirring the mixture until homogeneous, to provide an emulsion base, cooling the emulsion base to a temperature in the range of about 40 to about 50° C. and then adding the Compound (IV) ethosulfate containing ingredient to provide a lotion emulsion.

Direct Red 80 (Aldrich Chemical Co.) was used in place of rubine in a modification of the Rubine Dye Test. A dye solution was prepared by dissolving about 5 grams of Direct Red 80 in about 1000 mL of deionized water and adjusting the pH to about 3.5 with sulfuric acid. The substantivity test was performed as follows:

(a) A keratin substrate (wool cloth swatch, a hair swatch, or an area of human skin as described below) was first treated with a composition from Table 1 for a predetermined period of time, and then rinsed with deionized water;

(b) the so-treated substrate was then contacted with the dye solution for

a predetermined period of time and then rinsed with deionized water; and

(c) the relative amount of dye remaining on the substrate was determined visually and/or using a color meter; dye coloration remaining on the substrate indicates the presence of quaternized cosmetic agent, the degree of substantivity of the quaternized cosmetic agent is directly proportional to the relative amount of dye remaining on the substrate, as determined visually or by color density measurement.

Wool Substantivity: Separate 2-inch by 2-inch swatches of white wool cloth (worsted wool gabardine, Testafabrics, Inc.) were each soaked in one of Compositions A-D (in triplicate) for about 30 seconds and then rinsed for about 30 seconds in deionized water. The treated swatches were then each soaked for about 30 seconds in a large excess of dye solution (about 100 mL/swatch). The dye-soaked swatches were then rinsed with deionized water for about 30 seconds and allowed to dry at ambient room temperature. The intensity of the red coloration from the dye remaining on each swatch was determined using a MacBeth Color Eye 3100 with Optiview Lite version 1.9 software compared to a control wool swatch. The control wool swatch was soaked in a hydroalcoholic medium (9:1 parts by weight deionized water: isopropanol), then treated with the dye solution, and rinsed. The results of the calorimetric measurements are shown in Table 2. All of the swatches had a visible pink to red coloration. The most intensely colored swatches were those treated with Composition C, followed in order of decreasing intensity by those treated with Composition D, A, and B.

TABLE 2
Substantivity to Wool

| Treatment | MacBeth Redness Value |
|---|---|
| No Compound (IV) (control) | <1 |
| Composition A | 18 |
| Composition B | 12 |
| Composition C | 29 |
| Composition D | 23 |

Hair Substantivity: Swatches of virgin, natural brown, hair were obtained from International Hair Importers. Individual tresses were prepared, each about 6 inches (about 15.24 cm) in length, 1 inch (about 2.54 cm) in width, and about 2.75 g in weight. Each tress was sequentially bleached four times for about 30 minutes per bleaching, using a 6% hydrogen peroxide bleach solution adjusted to about pH 9 with ammonia hydroxide (about 250 g bleach solution/tress), rinsing with deionized water between each bleaching. The 4-times bleached tresses were then washed with a 15% sodium lauryl sulfate (SLS) solution, and rinsed with deionized water. The washed, bleached hair tresses were then allowed to dry at ambient room temperature.

Each individual bleached and dried hair tress was separately treated by soaking in one of Composition A, B, C or D (about 150 mL/tress) for about 2 minutes. Each treated hair tress was then rinsed with deionized water and then soaked, for about 30 seconds, in an excess (about 150 mL) of the dye solution. The tress was removed from the dye solution, rinsed with deionized water and allowed to dry. The color of each tress was then assessed visually in comparison to an undyed tress and to one another. Each of the tresses that was treated with a composition containing Compound (IV) ethosulfate exhibited a visually discernible red-orange to magenta color, indicating that at least a portion of the cationic Compound (IV) present in the compositions remained on the hair after rinsing. The hair treated with Composition C (about 0.5% active Compound (IV) ethosulfate) had the most intense color, followed, in decreasing intensity, by the tresses treated with Compositions A, D, and B. It is well known in the art and from experience that bleached hair, per se, retains substantially no dye in the absence of cationics.

Human Skin Substantivity: The forearms of one male volunteer subject were washed with a 15% SLS solution to remove any cosmetic agent present on the skin and rinsed. On each forearm, three roughly equal-sized, round areas (about 2 inches in diameter) were marked on the washed skin with a permanent marker. On one forearm about 0.5 mL of the emulsion Composition G was applied to each marked area and gently rubbed over the skin. The treated skin area was then rinsed, patted dry, and then about 3 drops of dye test solution was applied to each of the marked skin areas, gently rubbed in, and allowed to remain in contact with the skin for about 30 seconds. The dye-treated skin and forearm was then rinsed with water, blotted dry, and visually observed for dye coloration. The opposing forearm was treated in the same foregoing manner, except that the emulsion Composition H was used. A red coloration was visible on both forearms indicating cationic substantivity. Emulsion Composition G produced more visible coloration than emulsion Composition H. The result demonstrated that Compound (IV) ethosulfate was substantive to skin.

Numerous variations and modifications of the embodiments described above may be effected without departing from the spirit and scope of the novel features of the invention. No limitations with respect to the specific embodiments illustrated herein are intended or should be inferred.

1. A cosmetic agent comprising a cosmetically useful group bound to an amino functional group by a linking moiety;
the linking moiety comprising an alkylidene or an arylidene group bearing a first carboxyl group and a second carboxyl group;
the cosmetically useful group being bound to the first carboxyl group of the linking moiety;
the amino functional group comprising an amino substituent bound to a carbon atom of an alkylidene group bearing an oxygen substituent, the oxygen substituent forming an ester linkage with the second carboxyl group of the linking moiety.

2. A quaternized cosmetic agent comprising a compound of formula (I): 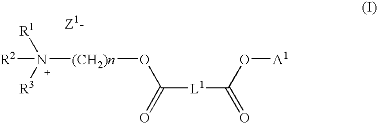
wherein R¹, R², and R³ are each independently selected from the group consisting of C₁-C₁₈ alkyl, C₁-C₁₈ alkenyl, and benzyl; n is an integer having a value in the range of 2 to about 18; L¹ is a divalent organic radical selected from the group consisting of C₁-C₁₈ alkylidene, C₆-C₁₀ arylidene and C₁-C₁₈ alkyl-substituted C₆-C₁₀ arylidene; Z¹- is selected from the group consisting of a halide ion, a C₁-C₁₈ alkylsulfate ion, a C₁-C₁₈ alkylsulfonate ion, a benzylsulfate ion, a benzylsulfonate ion, and an arylsulfonate ion; and A¹ is a physiologically tolerable, cosmetically useful group.

3. The quatemized cosmetic agent of claim 2 wherein A¹ is selected from the group consisting of an antioxidant, a vitamin, a provitamin, a coenzyme, an essential oil, a skin tanning agent, and a sunscreen agent.

4. The quaternized cosmetic agent of claim 2 wherein L¹ is a divalent organic radical selected from the group consisting of CH₂CH₂, CH₂CH₂CH₂, CH•CH, and 1,2-phenylene.

5. The quaternized cosmetic agent of claim 2 wherein R¹, R², and R³ are each independently selected from the group consisting of methyl, ethyl, and propyl.

6. The quaternized cosmetic agent of claim 2 wherein A¹ is a tocopherol moiety.

7. A method of preparing a quatemized cosmetic agent of claim 2 comprising the steps of:
(a) condensing an amino alcohol of formula (II) and a hydroxyl-substituted, physiologically tolerable, cosmetically useful material with a dicarboxylic acid or dicarboxylic anhydride to form a compound of formula (III): 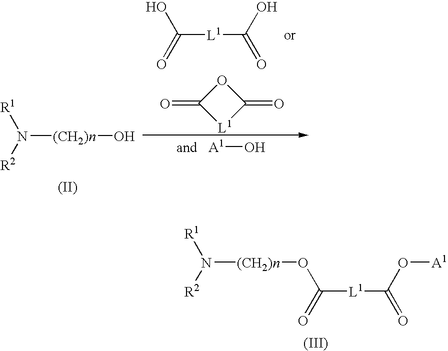
and
(b) quatemizing the compound of formula (III) with an alkylating agent R³-Z¹, to afford a compound of formula (I): 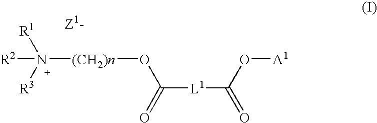
wherein in each of formulas (I), (II), and (III), R¹, R², and R³ are each independently selected from the group consisting of C₁-C₁₈ alkyl, C₁-C₁₈ alkenyl, and benzyl; n is an integer having a value in the range of 2 to about 18; L¹ is a divalent organic radical selected from the group consisting of C₁-C₁₈ alkylidene, C₆-C₁₀ arylidene and C₁-C₁₈ alkyl-substituted C₆-C₁₀ arylidene; Z¹- is selected from the group consisting of a halide ion, a C₁-C₁₈ alkylsulfate ion, a C₁-C₁₈ alkylsulfonate ion, a benzylsulfate ion, a benzylsulfonate ion, and an arylsulfonate ion; and A¹ is a physiologically tolerable, cosmetically useful group.

8. The method of claim 7 wherein the cosmetically useful material is selected from the group consisting of an antioxidant, a vitamin, a provitamin, a coenzyme, an essential oil, a skin tanning agent, and a sunscreen agent.

9. The method of claim 7 wherein L¹ is a divalent organic radical selected from the group consisting of CH₂CH₂, CH₂CH₂CH₂, CH=CH, and 1,2-phenylene.

10. The method of claim 7 wherein R¹, R², and R³ are each independently selected from the group consisting of methyl, ethyl, and propyl.

11. The method of claim 7 wherein the cosmetically useful material is alpha-tocopherol (vitamin E).

12. The method of claim 7 wherein the cosmetically useful material is first condensed with a dicarboxylic anhydride linking group to afford a product having a free carboxylic acid group, followed by condensation with the compound of formula (II) to produce the compound of formula (III).

13. A chemical compound having the formula (III): 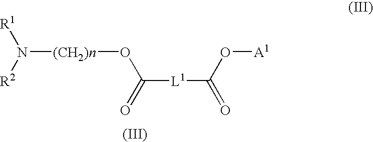
wherein R¹ and R² are each independently selected from the group consisting of C₁-C₁₈ alkyl, C₁-C₁₈ alkenyl, and benzyl; n is an integer having a value in the range of 2 to about 18; L¹ is a divalent organic radical selected from the group consisting of C₁-C₁₈ alkylidene, C₆-C₁₀ arylidene and C₁₋C₁₈ alkyl-substituted C₆-C₁₀ arylidene; and A¹ is a physiologically tolerable, cosmetically useful group.

14. A chemical compound having the formula (IV): 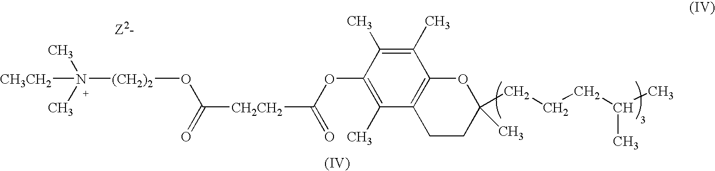
wherein Z²- is selected from the group consisting of a halide ion, a C₁-C₁₈ alkylsulfate ion, a C₁-C₁₈ alkylsulfonate ion a benzylsulfate ion, a benzylsulfonate ion, and an arylsulfonate ion

15. The compound of claim 14 wherein Z²- is a halide ion or a C₁-C₁₈ alkylsulfate ion.

16. The compound of claim 14 wherein Z²- is a chloride or bromide ion.

17. The compound of claim 14 wherein Z²- is an ethosulfate ion.

18. A composition comprising at least one cosmetic agent of claim 1.

19. The composition of claim 18 further comprising at least one solvent for the cosmetic agent.
